(19) United States
(12) Patent Application Publication
(10) Pub. No.: US 2009/0054876 A1
(43) Pub. Date: Feb. 26, 2009

(54) Compact ready-to-use urethral catheter assembly with means for injecting therapeutic liquid into urethral channel

(76) Inventors: German Borodulin, San Francisco, CA (US); Alexander Shkolnik, San Carlos, CA (US)

Correspondence Address: Alexander Shkolnik, 485 Dartmouth Avenue, San Carlos, CA, 94070, US

(21) Appl. No.: 11/895,079

(22) Filed: Aug. 24, 2007

(51) Int. Cl. A61M 27/00

(52) U.S. Cl. 604/544

(57) ABSTRACT

A catheter assembly suitable for intermittent catheterization comprising a cylindrical casing composed of two telescopically moveable parts one of which is connectable to and moveable with a piston which is rigidly connected to a urinary catheter. The interior of the cylindrical casing is filled with a lubricating, anesthetic, antiseptic liquid, or the like, which during catheterization is injected into the urethra and urinary bladder through the catheter under the effect of movement of the piston simultaneously with the insertion of the catheter into the urethra. The device is provided with an anti-infective cap which is inserted into the first third of the urethra and is used as a guide for inserting the catheter into the urethra without physical contact with the part if the urethra which is a harbor of infections.

### FIELD OF THE INVENTION

The invention relates to the field of urology, in particular to a compact ready-to-use catheter assembly with means for injection of a therapeutic liquid into the urethral channel. The catheter of the invention is a female catheter most suitable for intermittent self-catheterization.

### DESCRIPTION OF PRIOR ART

Intermittent catheterization may be necessary for patients who are unable to completely empty the urinary bladder during urination or unable to naturally urinate by themselves temporarily or permanently. Such a condition is known as “urinary retention.” Intermittent catheterization may also be required, e.g., to obtain a sterile urinary specimen from a patient in a doctor's office.

Intermittent catheterization involves the temporary placement of a catheter through the urethra to the bladder for emptying the latter. A goal of intermittent catheterization is to prevent urinary-tract infection by completely emptying the urinary bladder since accumulation of urine in the bladder over a long period of time may create conditions for development of a urinary-tract infection. After a bladder is effectively drained, urinary-tract infections cease to be a problem, and the kidneys are safeguarded.

Urinary catheters supplied for intermittent catheterization in general must have a lubricant applied to the outer surfaces thereof to facilitate insertion into the urethra. For example, hydrophilic urinary catheters have a hydrophilic outer surface coating that should be wetted with fluid such as water or a saline solution for a certain time period before insertion thereof into the urethra of a patient for lubrication purposes.

Various methods for lubricating urinary catheters have been previously proposed, examples of which are given below.

U.S. Pat. No. 5,209,726 makes known a self-lubricating urinary catheter and a method for self-catheterization. The catheter has an annular lubricant reservoir surrounding an inner tube having perforations. As the catheter is inserted into the urethra of a patient, the lubricant is forced from the reservoir into the inner tube and out and into the urethra through a discharge outlet in the catheter. Lubrication of the outer surface of the catheter, therefore, takes place only as the catheter is being inserted into the urethra of the patient, resulting in at least some initial discomfort for the patient on insertion of the catheter.

U.S. Pat. No. 3,967,728 makes known a catheter package comprising a urinary catheter and a rupturable, lubricant-containing pouch. One edge of the pouch is located within the package adjacent to the tip of the catheter, and the seal at that edge is constructed so that it ruptures when the pouch is squeezed in order to lubricate the tip of the catheter. Because only the tip of the catheter is lubricated, the patient still suffers discomfort on insertion of the catheter into the urethra.

U.S. Pat. No. 7,066,912 issued in 2006 to Nestenborg, et al., describes a wetting apparatus for wetting a hydrophilic urinary catheter comprising a wetting-fluid container that holds a wetting fluid and that can be opened by application of a pulling force thereto; a wetting receptacle; and a hydrophilic urinary catheter to be wetted by said wetting fluid and being arranged within said wetting receptacle. The wetting-fluid container is arranged within the wetting receptacle, and the wetting receptacle is extendable for opening the wetting container without rupturing the sealed portion of the wetting receptacle.

However, the devices described above are not available in a compact form, i.e., in a ready-to-use form, and therefore are inapplicable for self-catheterization and inconvenient for storage.

An example of a catheter that solves the above problems is a compact urinary prepackaged catheter developed by Coloplast Company (Denmark) and known under the trademark SpeediCath™, which can be obtained in a sterile and ready-to-use prehydrated form right out of the package. Each SpeediCath™ is prepackaged in sterile saline solution and in a plastic container that can be taken anywhere, similar to a ballpoint pen. Since SpeediCath™ is prelubricated, its coating is always optimally hydrated to be slippery and comfortable.

FIG. 1 is a longitudinal sectional view of the SpeediCath™ urethral catheter assembly for self-catheterization shown in a longitudinal section. The catheter assembly, which as a whole is designated by reference numeral 20, has a compact construction and consists of two casing parts 22 and 24 and a catheter unit 26 that is hermetically sealed inside the casing part 24. More specifically, the casing part 22 comprises a cylindrical sleeve with an open front end and a perforated bottom 28. The second casing part 24 comprises a tubular body, one end of which is closed and forms a handle 32 and a portion 34 which is opposite to the handle 32, is telescopically inserted into the open end of the casing part 22, and is closed by a holder 36 that holds a catheter 38 that extends in the axial direction X-X of the catheter assembly 20 into the interior of the second casing part 24 almost to the bottom of its central opening. The holder 36 has a first shoulder 39 (FIG. 1) and a second shoulder 40 to which the end face of the portion 34 of the second casing part 24 is sealed and strongly attached, e.g., by adhesion or thermal bonding so that a space 42 is formed among the inserted part of the holder 36, the outer surface of the catheter 38, and the inner surface of the second casing part 24. The area 37 of the sealed adhesive or bonding connection between the shoulder 40 of the holder 36 and the end face of the portion 34 is the area of subsequent separation of the second casing part 24 from the catheter unit formed by the portion 34, the holder 36, and the catheter 38, which will be described later. An annular groove 43 is formed between the shoulders 37 and 39.

In order to seal the space 42 in the closed state of the catheter assembly 20 shown in FIG. 1 and used for storage, the perforated bottom 28 of the casing part 22 has a small central plug 44 which is inserted into the rear opening of the holder that is connected to the central canal of the catheter 38, while the shoulder 40 of the holder 36 closes the perforations 46 and 48 in the bottom 28 of the casing part 22.

The surface of the catheter 38 has a hydrophilic coating and is preserved in an easy-to-insert prelubricated form by filling the sealed space 42 with a sterile physiological solution L in which the catheter 38 is retained during storage of the catheter assembly. Reference numeral 50 designates an opening in the side wall at the distal end of the catheter for discharge of urine into the interior of the catheter 38 and out from the catheter when the distal end of the catheter is inserted into the urinary bladder (not shown) for emptying the bladder through the catheter.

In the casing part 24, the portion 34 that is telescopically inserted into the open end of the casing part 22 has a diameter smaller than the remaining portion of the casing part 24 so that a shoulder 52 is formed on the casing part 24 that is butt contact with the mating end face 25 of the casing part 22. On a significant part of its length, the outer surface of the catheter assembly 20 is coated with a thin plastic film 54 for additional sealing of the area of the aforementioned contact in order to secure the connection.

For use, the plastic film 54 is torn off and removed. The casing part 22 is grasped by one hand, while the casing part 24 is pulled in the direction of arrow A (FIG. 1). Since the holder 36 is strongly attached by adhesion or thermal bonding to the end face of the portion 34 of the casing part 24 at the area 37, movement of the casing part 24 pulls the holder 36 in the same direction together with catheter, whereby the holder slides inside the casing part 22 in the direction of arrow A. This movement continues until the holder 36 reaches the end of the casing part 22 and assumes the position shown in FIG. 2. At this moment, a predeformed front edge 56 of the casing part 22 snaps into the annular groove 43 formed between the first shoulder 39 and a second shoulder 40 and is firmly secured to the casing 22.

The telescopic portion 34 of the casing part 24 is now withdrawn to its full length from the casing part 22. The user, while still grasping the casing part 22, now twists the casing part 24 with a force sufficient to disconnect the portion 34 from the shoulder 40 at the bonding area 37, whereby the condition shown in FIG. 2 is obtained. The disconnected casing part 24 is discarded, the interior of the space 42 is unsealed, and the liquid is poured out.

As a result, as shown in FIG. 3, the catheter 38 pretreated with the appropriate liquid is exposed, the casing part 22 forms a catheter handle convenient for manipulating the catheter 38, and the perforations 46 and 48 are opened for discharge of urine when the distal end of the catheter is inserted into the urinary bladder.

Although the SpeediCath™ catheter assembly described above provides a catheter in a ready-to-use form and in a sterile state, it does not guarantee prevention of infection that can be introduced into the urethra during insertion unless the urethral meatus has been thoroughly cleaned before catheterization. Unfortunately, means for cleaning the meatus are not always readily available under conditions wherein self-catheterization is performed.

A device that may partially solve the above-stated problem is a urinary plug for use in female patients described in U.S. Pat. No. 5,806,527 issued in 1998 to G. Borodulin, et al. Although the device is not intended for catheterization but rather for preventing involuntary release of urine from the bladder, the device is equipped with an infection-protective cap fitted on the distal end of the plug's tubular body insertable into the urinary bladder through the urethra. First, the infection-protective plug is inserted into the front infectious portion of the urethra, and then the plug is guided through the infection-protective cap without contacting the infectious part of the urethra.

A similar principle of protection against introduction of infection into the urinary bladder is used in a catheter for use in female patients for self-catheterization described in U.S. Pat. No. 6,544,240 issued in 2003 to Borodulin, et al. The device has a sterile infection-protective cap slidingly fitted onto the distal end of said catheter. The cap has a slit on its distal end for possibility of pushing the catheter through the sterile tip into the bladder without physical contact of the catheter with the infectious front part of the urethra.

However, the devices of U.S. Pat. No. 5,806,527 and U.S. Pat. No. 6,544,240 do not have any means that would pretreat the urethra or facilitate introduction of a plug or catheter into the bladder by treating the urethra during catheterization, e.g., by lubricating the urethral channel, etc. Furthermore, although the SpeediCath™ catheter assembly contains a sterile liquid, this liquid is intended only for treating the surface of the catheter and is not intended for treatment of the urethra.

In their earlier U.S. patent application Ser. No. ______, the applicants described a compact ready-to-use urethral catheter assembly suitable for intermittent self-catheterization which comprises a tubular body filled with a therapeutic liquid for introduction into the urethra. This liquid may comprise a lubricant, an anesthetic solution, an antiseptic solution, or a therapeutic solution to pretreat the urethra during catheterization. The device has a urinary catheter with a piston portion slidingly installed into the tubular body so that catheterization is accompanied by automatic injection of the therapeutic liquid into the urethra by means of the piston through a gap formed during catheterization between the outer surface of the catheter and the inner walls of the anti-infective cap which is used as a sterile guide for insertion of the catheter without contacting the initial part of the urethra, which may harbor infection. In addition to the above, the assembly is provided with built-in means for cleaning the urethral meatus before catheterization.

However, in the compact form in which it is stored, the catheter assembly described in U.S. patent application Ser. No. ______ is longer than the similar SpeediCath™ catheter assembly described above. The catheter is longer because in the aforementioned patent application, the proximal end of the plunger that is made integrally with the piston and catheter projects from the proximal end of the tubular body.

Another disadvantage of the SpeediCath™ catheter assembly relates its preparation for use, during which time a part of the tubular body is separated and must be discarded, while the remaining part is used for catheterization and is discarded after the procedure. Separate discarding of two parts of the SpeediCath™ catheter assembly is not always convenient, e.g., when a patient performs self-catheterization in other than her own home, etc. Moreover, in the SpeediCath™ catheter assembly, a thin coating film that must be removed prior to use of the device seals the area of separation of two parts of the tubular body. Provision of this film makes the device less convenient for use and spoils appearance of the assembly.

### OBJECTS AND SUMMARY OF THE INVENTION

It is an object of the present invention to provide a urethral catheter for self-catheterization that incorporates advantages of both the catheter assembly of U.S. patent application Ser. No. ______ and the SpeediCath™ catheter assembly. In other words, it is an object of the invention to provide a compact ready-to-use catheter for intermittent catheterization that is small in size, is provided with a sterile cap for guiding the catheter into the urethra without contacting the urethral walls in the first part thereof, and contains a therapeutic liquid that is automatically introduced into the urethra during catheterization. It is another object to provide a compact ready-to-use catheter for intermittent catheterization that can be conveniently discarded as a single indivisible piece after use. It is a further object to provide the aforementioned catheter assembly as a catheter assembly that does not require a coating film on the outer surface of its tubular body. Another object is to provide the aforementioned catheter with an adjustable mirror to facilitate the patient's ability to locate the position of the entrance into the urethra. A further object is to provide the aforementioned catheter assembly with a mechanism of locking for connecting the outer cylindrical casing to the inner tubular portion at the end of the piston stroke when the distal end of the catheter extends to its full length for employing the catheter assembly without the self-contained liquid and for using the interlocked outer and inner casing portions as an integral handle.

The urethral catheter of the invention consists essentially of an outer cylindrical casing, an inner tubular portion telescopically inserted into the outer cylindrical casing, a piston slidingly installed in the inner tubular portion and rigidly connected to the catheter, an anti-infective cap attached to the distal end of the outer cylindrical casing, a protective cover that covers the anti-infective cap in order to maintain its sterile condition, and a therapeutic liquid that fills the interior of the inner tubular portion between the piston and the anti-infective cap.

The aforementioned liquid may comprise an anesthetic liquid, lubricating liquid, antiseptic liquid, a physiological solution, or any other liquid that may be required for treating the urethra or the urethral bladder. The outer cylindrical casing has an open proximal end and a closed distal end with a central opening for passage of the catheter. On its proximal end, the outer cylindrical casing has an inwardly projecting edge.

The inner tubular portion has an elongated distal end that is telescopically and slidingly inserted into the outer cylindrical casing and a shorter and larger-in-diameter proximal or handle portion that is used as a handle that can be grasped by the user to move the inner tubular portion relative to the outer cylindrical casing. When the catheter assembly is in a compact or storage condition, a first shoulder that is formed on the front end of the handle portion is in butt contact with the aforementioned open proximal end of the outer cylindrical casing. The distal end of the inner tubular portion is open and has an inwardly projecting edge. In order to facilitate flexible deformation of this edge, it may be provided with short longitudinal cuts. The inner tubular portion also has on its outer surface a reduced-diameter portion that extends from the first shoulder to a second shoulder that is formed on the outer surface of the inner tubular portion at some distance from its distal end face. For convenience of grasping, the handle portion may have a flange.

The piston, which is slidingly installed in the inner tubular portion and is rigidly connected to the catheter, has a proximal or rear part that has a sliding fit inside the inner tubular portion, a distal or front part that has a diameter smaller than the rear part, and an annular groove between the rear part and the front part of the piston for snapping engagement with the aforementioned inwardly projecting edge on the distal end of the inner tubular portion. Where appropriate in the context of the present invention, the term “front” designates the distal end, and the term “rear” designates the proximal side or end of the catheter assembly or a respective part thereof.

A urethral catheter (that as a conventional one, comprises a thin tubular element with a length sufficient for passing through the patient's urethra to the urinary bladder) is rigidly attached to the front end of the piston, has a short front portion of a larger diameter and a long rear portion of a smaller diameter that extends from the front portion of the larger diameter to the front end face of the piston.

The anti-infective cap has a flange portion for attachment to the front end of the outer cylindrical casing and an axially extended cylindrical portion with a rounded front end having a length and diameter sufficient and suitable for insertion into the first third part of the urethra, which, as is known, can be a harbor of infection. The anti-infective cap has an inner cavity into which is inserted the aforementioned front portion of the catheter that has a larger diameter and that sealingly passes through the aforementioned opening formed in the closed front end of the outer cylindrical casing. As a result, in a closed, compact, and storage state, the urethra-treatment liquid that fills the interior of the inner tubular portion is sealed in a space defined by the inner surface of the inner tubular portion, the outer surface of the catheter, the front catheter portion of a larger diameter, and the front end face of the piston. The large-diameter portion of the catheter has a transverse opening for release of urine from the urinary bladder that communicates with the axial channel that passes from the aforementioned transverse opening through the catheter, the piston portion, and further through the open rear end of the handle portion for release of urine to the outside the catheter assembly. If necessary, the rear end of the handle portion may have a tubular extension to connect the catheter assembly to a conventional urine-collecting bag, etc.

The front, rounded end of the anti-infective cap has a slit for passing the catheter into the urethra. The front end of the outer cylindrical casing has an external thread for attaching the protective cover, which is screwed onto the aforementioned external thread to protect the anti-infective cap from contamination and to maintain its sterile condition. The sterile anti-infective cap can be prelubricated.

If necessary, the catheter assembly of the invention may be provided with a mirror device that may facilitate finding of the urethral opening. The mirror device may comprise a U-shaped body with a mirrored inner surface for folding onto the outer surface of the outer cylindrical casing and spring-loaded legs that can be snapped in the longitudinal slot formed on the external surface of the outer cylindrical casing with the possibility of pivotally adjusting the angular position of the mirror and moving the mirror in the longitudinal direction to find the position most convenient for observation. If necessary, the mirror device may have a flat mirror portion that can be intended for multiple uses, with the rest of the catheter assembly being disposable.

In order to prepare the catheter assembly for catheterization, the catheter assembly is converted from the catheter-hidden, or storage condition, to the working, or catheter-extended condition. For this purpose, the user, who may be a patient or one of the medical personnel, grasps the outer cylindrical casing with one hand, grasps the handle portion of the inner tubular portion with the other hand, and pulls out the inner tubular portion away from the outer cylindrical casing until the inwardly projecting edge on the front end of the inner tubular portion snaps into the annular groove formed on the outer surface of the piston. As a result, the piston and catheter become integrally connected to the inner tubular portion. In this condition, the urethra-treatment liquid remains sealed in the interior of the outer cylindrical casing between the front end of the outer cylindrical casing, the large-diameter portion of the catheter, the outer surface of the reduced-diameter portion of the catheter, the front end face of the piston, and the front end face of the inner tubular portion.

For catheterization, the protective cover is disconnected from the outer cylindrical casing, and the anti-infective cap is inserted into the urethra, if necessary, with the use of the mirror device. As in the above condition, the catheter is rigidly connected to the assembly of the piston with the inner tubular portion, the latter is shifted forward relative to the outer cylindrical casing so that the piston slides inside the outer cylindrical casing and displaces the liquid into the urethra and further to the bladder through the gap that is now formed between the outer surface of the small-diameter portion of the catheter and the inner walls of the central opening in the anti-infective cap. The liquid flows directly to the urethra since the front end of the flange of the anti-infective cap is tightly pressed against the area of the body around the urethral meatus (the entrance into the urethra). At the end of the piston stroke, the catheter is extended to its extreme outer position, in which the urine-release opening provided on the large-diameter portion is located inside the urinary bladder. This allows empting of the bladder. Thus, introduction of the catheter into the urethra occurs simultaneously with injection of the therapeutic liquid that can be used as a lubricant, anesthetic liquid, antiseptic liquid, or the like.

### BRIEF DESCRIPTION OF THE DRAWINGS

FIG. 1 is a longitudinal sectional view of a known urethral catheter assembly for intermittent catheterization in the closed and compact state.

FIG. 2 is a longitudinal sectional view of the urethral catheter of FIG. 1 in the condition wherein the first part of the casing is separated from the second part, and a predeformed front edge of the second part snaps into the annular groove of the catheter holder.

FIG. 3 is a longitudinal sectional view of the catheter of FIG. 1, with the catheter extended from the casing and ready for use.

FIG. 4 is a longitudinal sectional view of the urethral catheter assembly of the present invention in the closed and compact state with the urethra-treating liquid sealed inside the catheter casing.

FIG. 5 is a longitudinal sectional view of the urethral catheter of FIG. 4 in the condition wherein the inner tubular portion is withdrawn and the edge of the outer casing snaps into the grooves of the piston.

FIG. 6 is a longitudinal sectional view of the catheter assembly of FIG. 4 during catheterization, with the catheter inserted into the urethra in a fully extended position.

FIG. 7 is a three-dimensional view of the distal end of the catheter of the invention equipped with a mirror in the working position.

FIG. 8 is a side view of the distal end of the catheter of FIG. 7 with the mirror in the folded position.

FIG. 9 is a sectional view of the proximal part of the catheter assembly of the invention that shows a tubular extension for connection of the catheter assembly to a urine collector.

FIG. 10 is a three-dimensional view illustrating a modification of the catheter assembly of the invention which is provided with a mechanism for locking the outer cylindrical casing to the inner tubular portion at the end of the piston stroke when the distal end of the catheter extends to its full length.

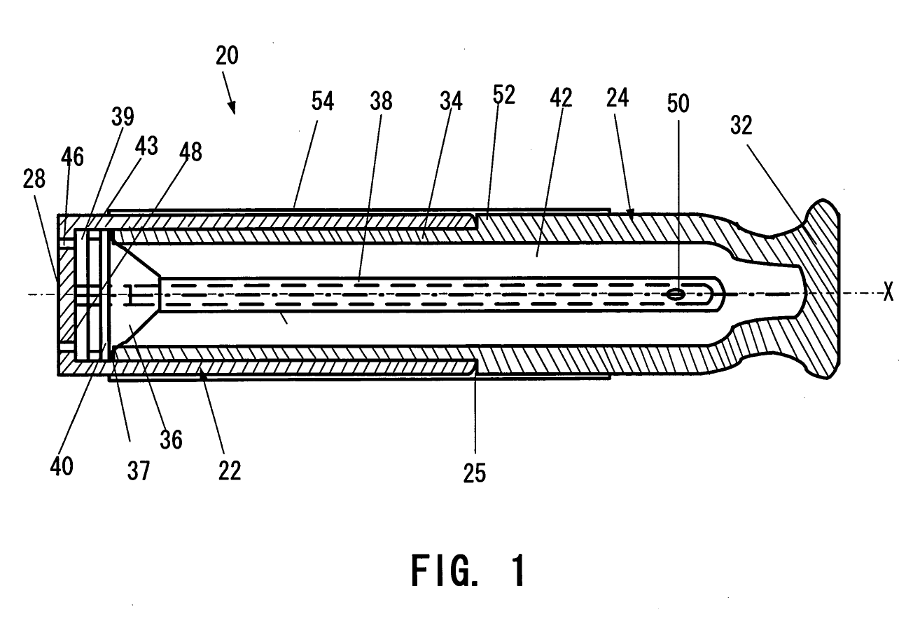

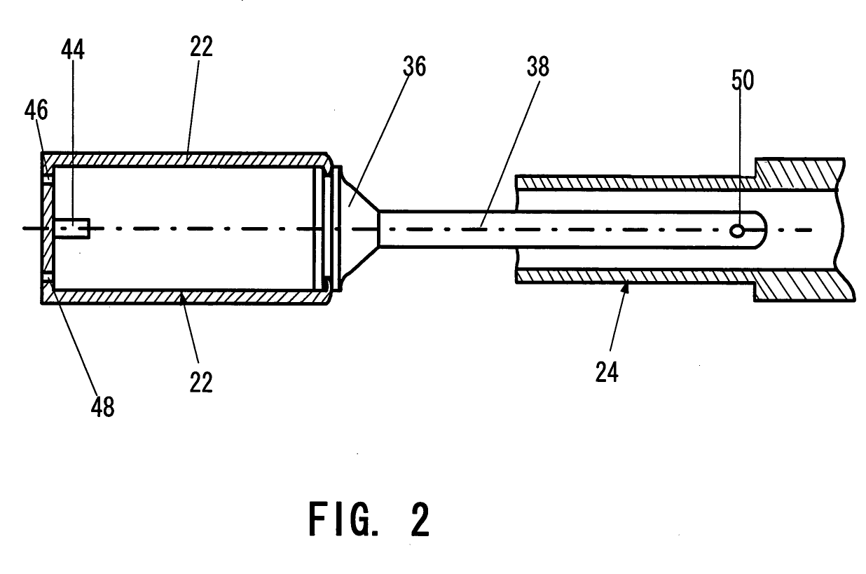

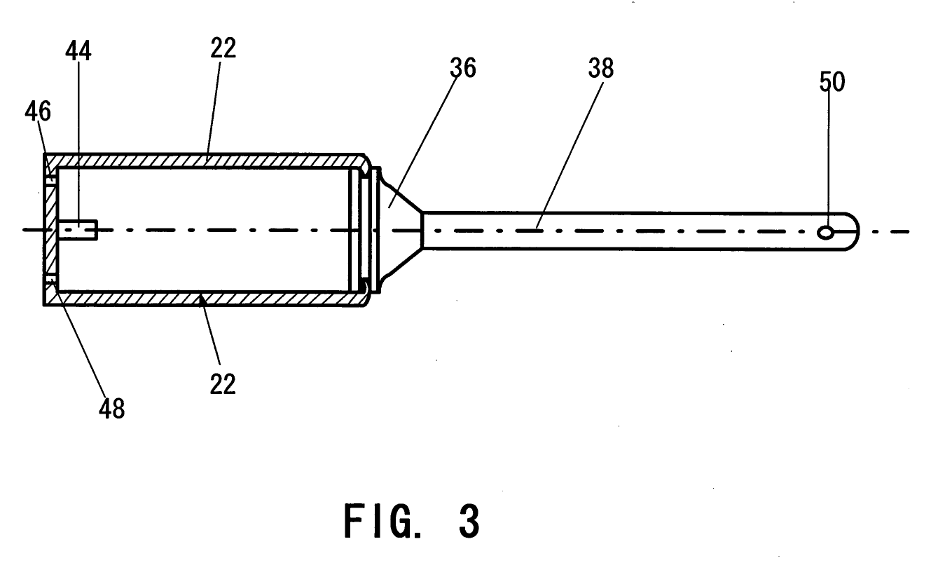

### DETAILED DESCRIPTION OF THE INVENTION

The invention will now be described in more detail with reference to accompanying drawings.

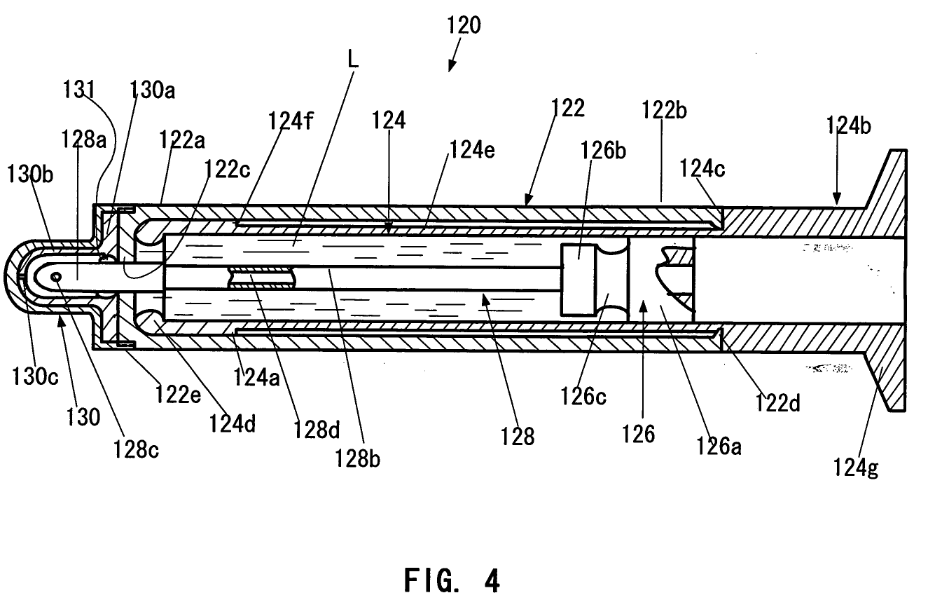

FIG. 4 is a longitudinal sectional view of the urethral catheter assembly of the present invention which in general is designated by reference numeral 120 and is shown in a closed and compact state in which is it stored with a therapeutic liquid L sealed inside the urethral catheter assembly 120.

The urethral catheter 120 of the invention consists essentially of an outer cylindrical casing 122, an inner tubular portion 124 telescopically inserted into the outer cylindrical casing 122, a piston 126 slidingly installed in the inner tubular portion 124 and rigidly connected to the catheter 128, an anti-infective cap 130 attached to the distal end 122a of the outer cylindrical casing 122, a protective cover 132 that covers the anti-infective cap 130 in order to maintain its sterile condition, and the aforementioned therapeutic liquid L, which, in the storage state of the assembly shown in FIG. 4, fills the interior of the inner tubular portion 124 between the piston 126 and the anti-infective cap 130.

The aforementioned liquid L may comprise an anesthetic liquid, lubricating liquid, antiseptic liquid, a physiological solution, or any other liquid that may be required for treating the urethra or the urethral bladder. The outer cylindrical casing 122 has an open proximal end 122b, while the aforementioned distal end 122a is closed but has a central opening 122c for passage of the catheter 128. On its proximal end 122b, the outer cylindrical casing 122 has an inwardly projecting edge 122d.

The inner tubular portion has an elongated distal end 124a that is telescopically and slidingly inserted into the outer cylindrical casing 122 and a shorter and larger-in-diameter proximal or handle portion 124b that is used as a handle that can be grasped by the user to move the inner tubular portion 124 relative to the outer cylindrical casing 122.

When the catheter assembly 120 is in a compact or storage condition shown in FIG. 4, a first shoulder 124c that is formed on the front end of the handle portion 124b is in butt contact with the aforementioned open proximal end 122b of the outer cylindrical casing. The distal end 124a of the inner tubular portion is open and has an inwardly projecting edge 124d. In order to facilitate flexible deformation of this edge, it may be provided with short longitudinal cuts (not shown in the drawing). The inner tubular portion also has on its outer surface a reduced-diameter portion 124e that extends from the first shoulder 124c to a second shoulder 124f that is formed on the outer surface of the inner tubular portion 124 at some distance from the inwardly projecting edge 124d. For convenience of grasping, the handle portion 124b may have a flange 124g.

The piston 126, which is slidingly installed in the inner tubular portion 124 and is rigidly connected to the catheter 128, has a proximal or rear part 126a that has a sliding fit inside the inner tubular portion 124, a distal or front part 126b that has a diameter smaller than the rear part 126a, and an annular groove 126c between the rear part 126a and the front part 126b of the piston for snapping engagement with the aforementioned inwardly projecting edge 124d on the distal end of the inner tubular portion 124. Where appropriate in the context of the present invention, the term “front” will designate the distal end, and the term “rear” will designate the proximal side or end of the catheter assembly 120 or a respective part thereof (FIG. 4).

A urethral catheter 128 (that as a conventional one, comprises a thin tubular element with a length sufficient for passing through the patient's urethra to the urinary bladder) is rigidly attached to the front end of the piston 126, has a short front portion 128a of a larger diameter and a long rear portion 128b of a smaller diameter that extends from the front portion 128a of the larger diameter to the front end face of the piston 126 (FIG. 4).

The anti-infective cap 130 has a flange portion 130a for attachment to the front end 122a of the outer cylindrical casing 122 and an axially extended cylindrical portion 130b (FIG. 4) with a rounded front end having a length and diameter sufficient and suitable for insertion into the first third part of the urethra, which, as is known, can be a harbor of infection. The anti-infective cap has an inner cavity inserted into which is the aforementioned front portion 128a of the catheter 128 that has a larger diameter and that sealingly passes through the aforementioned opening 122c formed in the closed front end 122a of the outer cylindrical casing 122. As a result, in a closed, compact, or storage state, the urethra-treatment liquid L that fills the interior of the inner tubular portion 124 is sealed in a space defined by the inner surface of the inner tubular portion 124, the outer surface of the catheter 128, the front catheter portion 128a of a larger diameter, and the front end face of the piston 126. The large-diameter portion 128a of the catheter has a transverse opening 128c for release of urine from the urinary bladder that communicates with the axial channel 128d (FIG. 4) that passes from the aforementioned transverse opening through the catheter, the piston portion, and further through the open rear end of the handle portion 124b for release of urine to the outside the catheter assembly 120.

The front, rounded end on the cylindrical part 130b of the anti-infective cap 130 has a slit 130c (FIG. 4) for passing the catheter 128 into the urethra. The front end 122a of the outer cylindrical casing 122 has an external thread 122e for attaching the protective cover 132, which is screwed onto the aforementioned external thread 122e to protect the anti-infective cap 130 from contamination and to maintain its sterile condition. The sterile anti-infective cap 130 can be prelubricated.

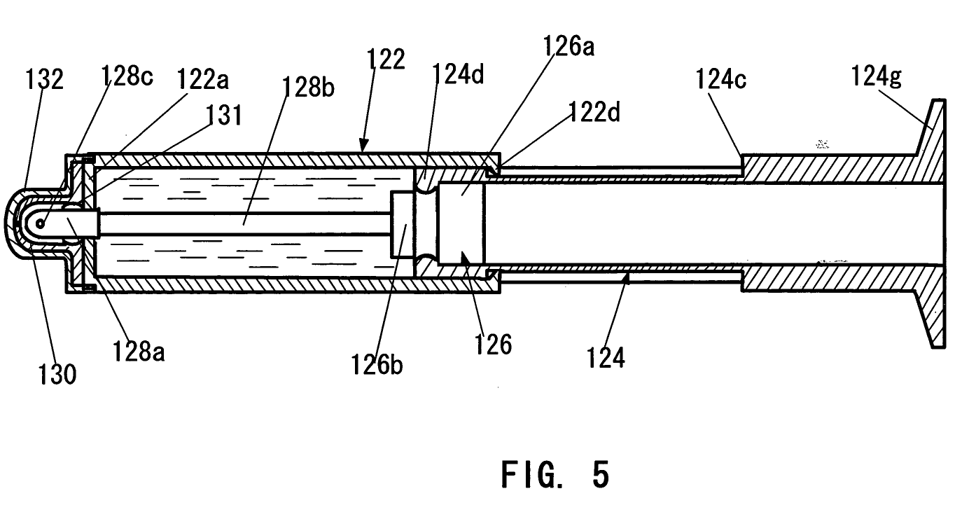

In order to prepare the catheter assembly 120 for catheterization, the catheter assembly 120 is converted from the catheter-hidden, or storage condition shown in FIG. 4, to the extended position shown in FIG. 5 ready for insertion into the urethra and for injection of the liquid L into the urethra and urinary bladder. For this purpose, the user, who may be a patient or one of the medical personnel, grasps the outer cylindrical casing 122 with one hand, grasps the handle portion 124b of the inner tubular portion 124 with the other hand, and pulls out the inner tubular portion 124 away from the outer cylindrical casing 122 until the inwardly projecting edge 124d on the front end of the inner tubular portion 124 snaps into the annular groove 124c formed on the outer surface of the piston 126. As a result, the piston 126 and the catheter 128 become integrally connected to the inner tubular portion 124. In this condition, the urethra-treatment liquid L remains sealed in the interior of the outer cylindrical casing 122 between the front end 122a of the outer cylindrical casing 122, the large-diameter portion 128a of the catheter 128, the outer surface of the reduced-diameter portion 128b of the catheter 128, the front end face of the piston part 126b, and the front end face of the inner tubular portion 124.

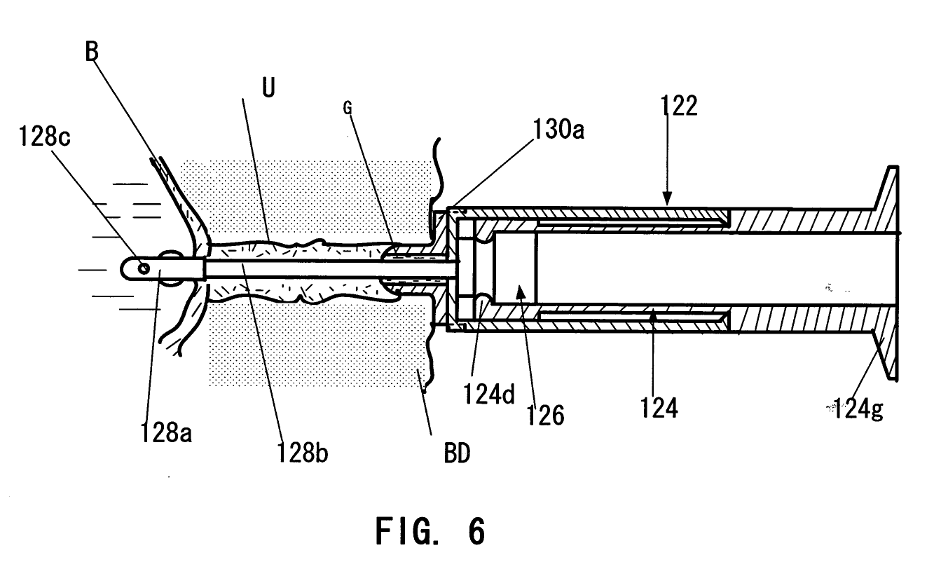

For catheterization, the protective cover 132 is disconnected from the outer cylindrical casing 122, and the anti-infective cap 130 is inserted into the urethra (as shown in FIG. 6). As the catheter 128 is rigidly connected to the assembly of the piston 126 with the inner tubular portion 124, the latter is shifted forward relative to the outer cylindrical casing 122 so that the piston 126 slides inside the outer cylindrical casing 122 and displaces the liquid L into the urethra U and further to the urinary bladder B (FIG. 6) through the gap G that is now formed between the outer surface of the small-diameter portion 128b of the catheter 128 and the inner walls of the central opening in the anti-infective cap 130. The liquid L flows directly to the urethra U since the front end of the flange 130a of the anti-infective cap 130 is tightly pressed against the area of the body BD around the urethral meatus (the entrance into the urethra). At the end of the piston stroke, the catheter 128 is extended to its extreme outer position shown in FIG. 6, in which the urine-release opening 128c provided on the large-diameter portion 128a is located inside the urinary bladder B. This allows empting of the bladder B. Thus, introduction of the catheter 128 into the urethra U occurs simultaneously with injection of the therapeutic liquid L that can be used as a lubricant, anesthetic liquid, antiseptic liquid, or the like.

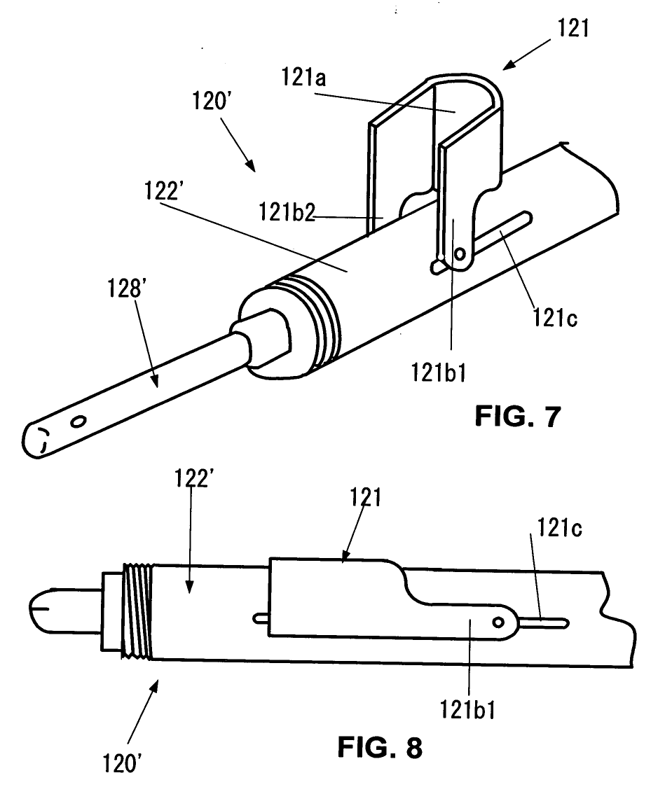

If necessary, as shown in FIGS. 7 and 8, the catheter assembly 120′ of the invention may be provided with a mirror device 121 that may facilitate finding of the urethral opening. The mirror device 121 may comprise a U-shaped body with a mirrored inner surface 121a for folding onto the outer surface of the outer cylindrical casing 122′ and spring-loaded legs 121b1 and 121b2 that can be snapped into the longitudinal slots (only one of which 121c is shown in FIGS. 7 and 8) formed on the external surface of the outer cylindrical casing 122′ with the possibility of pivotally adjusting the angular position of the mirror 121 and moving the mirror 121 in the longitudinal direction along the slots to find the position most convenient for observation. If necessary, the mirror device 121 may have a flat mirror portion (not shown) that can be intended for multiple uses, with the rest of the catheter assembly being disposable. Reference numeral 128′ designates the catheter.

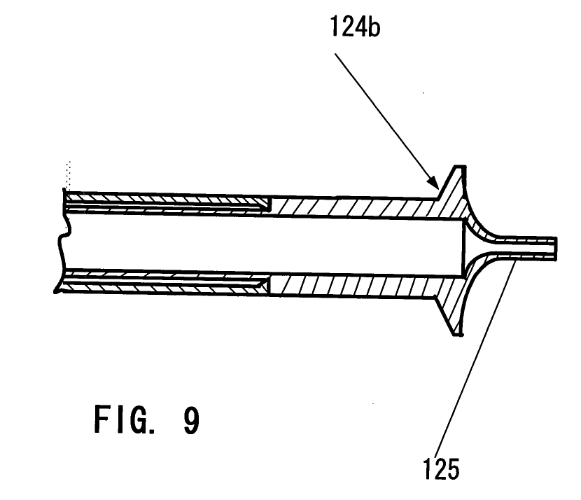

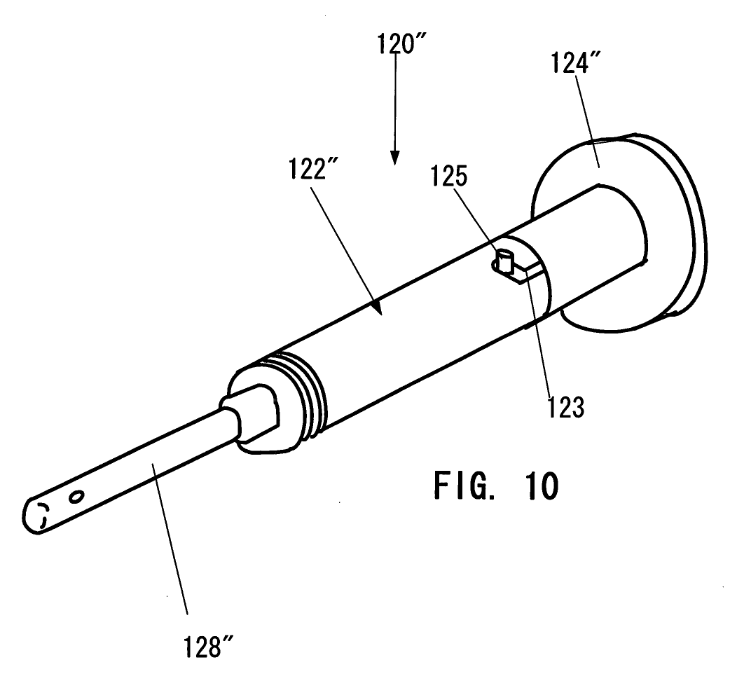

As shown in FIG. 9, the rear end of the handle portion 124b may have a tubular extension 125 for connection of the catheter assembly to a conventional urine-collecting bag, etc.

FIG. 10 shows a modification of the catheter assembly 120″ which is provided with means for locking the outer cylindrical casing 122″ to the inner tubular portion 124″ in the extended condition of the catheter 128″ shown in FIGS. 6 and 10 for using the extended catheter 128″ as a conventional one and for using the outer cylindrical casing 122″ interlocked with the inner tubular portion 124″ as a handle. In this case, when the urinary catheter 128″ is moved forward, the liquid is expelled from the interior of the catheter assembly onto the outer surface of the urinary catheter 128″ and pre-lubricate the latter so that after urinary catheter is shifted into the most extended position, the outer cylindrical casing 122″ is fixed to the inner tubular portion 124″, and the pre-lubricated catheter can be used for catheterization while the fixed parts of the casing can be used as a handle.

The aforementioned locking means comprises a bayonet-type lock formed by an L-shaped slot 123 formed on the proximal end of the outer cylindrical casing and a pin 125 projecting radially outward from the surface of the inner tubular portion 124″ just in front of the handle portion 124b″.

If necessary, the construction shown in FIG. 10 makes it possible to use the catheter assembly without filling the interior of the outer cylindrical casing 122″ with therapeutic liquid L. When the urinary catheter 128″ is extended to its full length, it is pre-lubricated, and then is used for catheterization.

Thus it has been shown that the present invention provides a urethral catheter assembly for self-catheterization that has a compact ready-to-use construction, is suitable for intermittent catheterization, is small in size, is provided with a sterile cap for guiding the catheter into the urethra without contacting the urethral walls in the first part thereof, and contains a therapeutic liquid that is automatically introduced into the urethra during catheterization. The catheter assembly of the invention can be conveniently discarded as a single indivisible piece after use, does not require a coating film on the outer surface of its tubular body, and can be equipped with an adjustable mirror to facilitate the patient's ability to locate the position of the entrance into the urethra.

1. A catheter assembly comprising:
a hollow housing having an interior and composed of a first part and a second part which is telescopically and slidingly inserted into the first part, the first part and the second part forming an interior;
a urinary catheter for insertion into the urethra during catheterization;
connection means for connection of the urinary catheter to the second part for joint movement therewith when the urinary catheter is extended from the hollow housing to a length suitable for catheterization, the urinary catheter having a distal part and a proximal part; and
an anti-infective cap for insertion of the urinary catheter therethrough into the urethra.

2. The catheter assembly of claim 1, further provided with a mechanism for rigidly connecting the first part to the second part of the hollow housing when the urinary catheter is connected to the second part and is extended from the hollow housing to the length suitable for catheterization.

3. The catheter assembly of claim 2, wherein the mechanism for rigidly connecting the first part to the second part is an L-shaped slot on the end of one of the first part and the second part and a pin insertable into the L-shaped slot formed on the other of the first part and the second part so that both parts can be connected in a manner of a bayonet connection.

4. The catheter assembly of claim 2, further comprising a therapeutic liquid that fills the aforementioned interior; and
a piston rigidly connected to the proximal part of the urinary catheter for displacement of the therapeutic liquid from the aforementioned interior simultaneously with the aforementioned joint movement of the second part together with the piston and the urinary catheter through the anti-infective cap.

5. The catheter assembly of claim 4, wherein the anti-infective cap is sterile and the catheter assembly is further provided with a protective cap that is fitted onto the anti-infective cap and is attachable to the hollow housing.

6. A catheter assembly for urinary catheterization comprising:
an outer cylindrical casing having a distal end and a proximal end;
an inner tubular portion having a distal end, a proximal end, and telescopically inserted into aforementioned outer cylindrical casing,
a piston slidingly installed in the inner tubular portion;
a urinary catheter located inside the inner tubular portion, connected to the piston and passing therethrough, said catheter having a distal end and a proximal end;
an anti-infective cap attached to the distal end of the outer cylindrical casing and having an inner surface, an interior, and a slit for passing the urinary catheter, the distal end of the urinary catheter being sealingly inserted into the interior of the anti-infective cap; and
a therapeutic liquid that is contained in the inner tubular portion;
the catheter assembly having a first condition in which the inner tubular portion is completely inserted into the outer cylindrical casing, and a second condition in which the inner tubular portion is withdrawn from outer cylindrical casing through the aforementioned proximal end of the outer cylindrical casing;
the catheter assembly being further provided with a mechanism for locking the piston to the distal end of the inner tubular portion when the inner tubular portion assumes the aforementioned second condition;
the therapeutic liquid being sealed inside the catheter assembly in the first condition and in the second condition but is displaced from the catheter assembly through the slit of the anti-infective cap when in said second condition the inner tubular portion is moved together with the piston toward the anti-infective cap.

7. The catheter assembly of claim 6, wherein the mechanism for locking the piston to the distal end of the inner tubular portion when the inner tubular portion assumes the aforementioned second condition comprises an inwardly projecting edge on the distal end of the inner tubular portion and an annular groove on the periphery of the piston for snapping connection with the aforementioned inwardly projecting edge.

8. The catheter assembly of claim 7, wherein the distal end of the urinary catheter has a portion of an increased diameter, and the remaining part of the urinary catheter that extends from the portion of the increased diameter to the piston comprises a portion of a reduced diameter which is smaller than the diameter of the aforementioned inner surface of the anti-infective cap, so that when the urinary catheter is pushed forward and extends from the anti-infective cap, a gap is formed between the portion of a reduced diameter of the catheter and the inner surface of the anti-infective cap for passage of the therapeutic liquid out from the catheter assembly.

9. The catheter assembly of claim 6, wherein the anti-infective cap is sterile, and the catheter assembly is further comprising a protective cover that can be fitted onto the anti-infective cap.

10. The catheter assembly of claim 9, wherein the distal end of the outer cylindrical casing has means for attaching the protective cover when the protective cover is fitted onto the anti-infective cap.

11. The catheter assembly of claim 8, wherein the anti-infective cap is sterile, and the catheter assembly is further comprising a protective cover that can be fitted onto the anti-infective cap.

12. The catheter assembly of claim 11, wherein the distal end of the outer cylindrical casing has means for attaching the protective cover when the protective cover is fitted onto the anti-infective cap.

13. The catheter assembly of claim 6, wherein the inner tubular portion has a handle portion, which is formed on the proximal end of the inner tubular portion; the handle portion having a distal end and a first shoulder formed on the distal end of the handle portion, said first shoulder being in a butt contact with the proximal end of the outer cylindrical casing when the catheter assembly is in the first condition; the inner tubular portion having and outer surface and a second shoulder formed on the outer surface thereof near the distal end of the inner tubular portion; the proximal end of the outer cylindrical casing having an inwardly projecting edge that engages with the second shoulder of the inner tubular portion when the catheter assembly is in the aforementioned second condition.

14. The catheter assembly of claim 8, wherein the inner tubular portion has a handle portion, which is formed on the proximal end of the inner tubular portion; the handle portion having a distal end and a first shoulder formed on the distal end of the handle portion, said first shoulder being in a butt contact with the proximal end of the outer cylindrical casing when the catheter assembly is in the first condition; the inner tubular portion having and outer surface and a second shoulder formed on the outer surface thereof near the distal end of the inner tubular portion; the proximal end of the outer cylindrical casing having an inwardly projecting edge that engages with the second shoulder of the inner tubular portion when the catheter assembly is in the aforementioned second condition.

15. The catheter assembly of claim 8, wherein the anti-infective cap is sterile, and the catheter assembly is further comprising a protective cover that can be fitted onto the anti-infective cap.

16. The catheter assembly of claim 15, wherein the distal end of the outer cylindrical casing has means for attaching the protective cover when the protective cover is fitted onto the anti-infective cap.

17. The catheter assembly of claim 6, wherein the therapeutic liquid is selected from a lubricating liquid, anesthetic liquid, and antiseptic liquid.

18. The catheter assembly of claim 8, wherein the therapeutic liquid is selected from a lubricating liquid, anesthetic liquid, and antiseptic liquid.

19. The catheter assembly of claim 11, wherein the therapeutic liquid is selected from a lubricating liquid, anesthetic liquid, and antiseptic liquid.

20. The catheter assembly of claim 13, wherein the therapeutic liquid is selected from a lubricating liquid, anesthetic liquid, and antiseptic liquid.
